(19) United States
(12) Patent Application Publication
(10) Pub. No.: US 2014/0302180 A1
(43) Pub. Date: Oct. 9, 2014

(54) COMPOSITION COMPRISING CHICORY EXTRACT

(71) Applicant: DIALPHA, Montferrier Sur Lez (FR)

(72) Inventors: Nicolas Chapal, Combaillaux (FR); Vickram Beejmohun, Montpellier (FR)

(21) Appl. No.: 14/359,008

(22) PCT Filed: Nov. 19, 2012
(86) PCT No.: PCT/EP2012/072984

§ 371 Date: May 16, 2014

(30) Foreign Application Priority Data
Nov. 18, 2011 (EP) 11306518.9

(51) Int. Cl. A61K 36/28

(52) U.S. Cl. CPC A61K 36/28
USPC 424/725

(57) ABSTRACT

The present invention relates to a chicory extract for allowing reduction or control of body weight or limitation of body weight gain, reduction or limitation of increase of fat storage, of fatty liver, of liver triglycerides level, of hypertriglyceridemia, of glycemia level, of insulinemia level, of insulin resistance, and/or of different factors of metabolic syndrome, and to a composition comprising such an extract.

The present invention relates to a chicory extract for allowing reduction or control of body weight or limitation of body weight gain, reduction or limitation of increase of fat storage, of fatty liver, of liver triglycerides level, of hypertriglyceridemia, of glycemia level, of insulinemia level, of insulin resistance, of pre-diabetes and/or of different factors of metabolic syndrome, and to a composition comprising such an extract.

More particularly, the extract may exhibit an action on risk factor(s) which may have an impact on fatty cirrhosis, cardiovascular disease, diabetes complications and/or diabetes. Among these risk factors, may be cited the overweight, in particular obesity, the increasing of fat storage, of fatty liver, of liver triglycerides level, of hypertriglyceridemia, of glycemia level, of insulinemia level, of insulin resistance and/or of different factors of metabolic syndrome.

The invention concerns also food or dietary supplement comprising such an extract.

The invention also relates to a composition for use for prevention or treatment of one or several factors of metabolic syndrome, obesity, fatty cirrhosis, cardiovascular disease, diabetes complications and/or diabetes.

### BACKGROUND OF THE INVENTION

The modern society leads more and more to the increase of risk factors of diseases such as fatty cirrhosis, cardiovascular disease, diabetes complications and/or diabetes.

The known compounds or compositions which may be used to treat these diseases and/or to limit one or several of their risk factors, in particular the reduction of body weight, may be insufficiently efficient, too expensive, exhibiting undesired side effects, having insufficient organoleptic qualities, they may change the colour, taste, and/or aspect of the food it is intended to be combined with, difficult to introduce into food, at least with some types of food, insufficiently stable, may present a low solubility, may be insufficiently versatile, may present a sourcing not stable enough, and/or abundant, or they may come from a precursor or precursors which may have other uses.

On the other hand the diets for regulating body weight often have a limited success. Low caloric diets for example may cause a temporary loss of body weight but have not proven their efficiency on the long term for people wanting to lose weight and to maintain a defined weight.

The invention thus aims to solve all or part of the above cited problems.

### GENERAL DESCRIPTION OF THE INVENTION

According to an aspect, the invention has for subject matter an extract from a plant of the Cichorium genus comprising phenol derivatives, including the three major phenolic acids derivatives identified as chicoric acid, caftaric acid and chlorogenic acid and including flavonoids of Quercetin, and Luteolin derivatives.

The invention may show all or part of the following advantages:
very low cost, furthermore it may value a part of a plant which is not used until now, in particular the leaves, but which is easily collected,
strong biological effect,
food grade quality of the extract, thus leading to the possibility of a use as preventive agent (as it has no or very little toxicity), and/or
easiness to obtain an active agent.

Other advantages will be shown in the following description.

According to another aspect, the invention has for subject matter a composition comprising an extract of a plant of the Cichorium genus comprising phenolic acids derivatives and flavonoids derivatives, and optionally a carrier.

The extract or the composition comprising the extract may be intended for allowing reduction or control of body weight or limitation of body weight gain, reduction or limitation of increasing of fat storage, of fatty liver, of liver triglycerides level, of hypertriglyceridemia, of hyperglycemia of glycemia level, of insulinemia, of insulin resistance, pre-diabetes and/or of different factors of metabolic syndrome.

The extract or the composition comprising the extract may be used in a curative or prophylactic treatment of control of body weight, and/or of weight gain, and/or of increased fat storage, and/or of fatty liver, and/or of high liver triglycerides level, of pre-diabetes and/or of hypertriglyceridemia, and/or for hyperglycemia, and/or of high glycemia level, and/or of hyperinsulinemia, and/or of insulin resistance, and/or of metabolic syndrome

The invention also has for subject matter a food composition, solid or liquid, comprising an extract of a plant of the Cichorium genus comprising phenolic acids derivatives and flavonoids derivatives, and optionally a carrier.

The invention also has for subject matter a dietary or food supplement or a nutraceutical or food composition comprising an extract of a plant of the Cichorium genus comprising phenolic acids derivatives and flavonoids derivatives, and optionally a carrier, in particular for allowing reduction of body weight or limitation of body weight gain, reduction or limitation of increasing of fat storage, of fatty liver, of liver triglycerides level, of hypertriglyceridemia, of glycemia level, of insulinemia level, of insulin resistance, and/or of one, several or all the factors of metabolic syndrome.

According to another aspect, the invention has for subject matter a pharmaceutical composition comprising an extract of a plant of the Cichorium genus comprising phenolic acids derivatives and flavonoids derivatives, and optionally a carrier, in particular for use for preventing and/or treating fatty cirrhosis, cardiovascular disease, diabetes complications and/or diabetes and/or one or several factors of metabolic syndrome, for example high level of insulin, hypercholesterolimia, hypertension, overweight, in particular obesity, and hyperglycemia.

The invention also concerns a method of preventing and/or treating fatty cirrhosis, cardiovascular disease, diabetes complications and/or diabetes and/or one or several factors of metabolic syndrome, for example high level of insulin, hypercholesterolimia, hypertension, overweight, in particular obesity, and hyperglycemia in a subject in need thereof, comprising administering to said subject a composition comprising an extract of a plant of the Cichorium genus according to the invention.

The composition may be used for modulating body weight of a subject, in particular losing, controlling or helping to control the body weight of a subject.

The invention also concerns a method for modulating body weight of a subject, in particular losing, controlling or helping to control the body weight of a subject in need thereof, comprising administering to said subject a composition comprising an extract of a plant of the Cichorium genus according to the invention.

In the instant description a subject may be human or animal, and in particular mammal.

According to still another aspect, the invention has for subject matter the use of an extract of a plant of the Cichorium genus comprising phenolic acids derivatives and flavonoids derivatives for the preparation of a medicament or of a pharmaceutical composition.

Following yet another aspect, the invention has for subject matter a diet, in particular for allowing reduction of body weight or limitation of body weight gain, limitation or reduction increase of fat storage, of fatty liver, of liver triglycerides level, of hypertriglyceridemia, of glycemia level, of insulinemia, of insulin resistance, and/or of different factors of metabolic syndrome, comprising the step of at least one daily taking of a composition comprising an extract of a plant of the Cichorium genus comprising phenolic acids derivatives and flavonoids derivatives.

The invention also concerns a method for limiting or reducing the increase of fat storage, of fatty liver, of liver triglycerides level, of hypertriglyceridemia, of glycemia level, of insulinemia, of insulin resistance, and/or of different factors of metabolic syndrome in a subject in need thereof, comprising administering to said subject a composition comprising an extract of a plant of the Cichorium genus according to the invention.

### BRIEF DESCRIPTION OF THE FIGURES

FIG. 1 shows HPLC chromatograms of hydro-alcoholic plant extracts at 280 nm

FIG. 2 shows the effect of chicory extracts of the invention on blood glucose in obese pre-diabetic mouse.

FIG. 3 presents the effect of chicory extracts of the invention on blood insulin and insulin resistance in obese pre-diabetic mouse.

FIG. 4 shows the effect of chicory extracts of the invention on body weight and fat storage in obese pre-diabetic mouse.

FIG. 5 presents the effect of chicory extracts of the invention on fatty liver or hepatic steatosis in obese pre-diabetic mouse.

FIG. 6 shows the dose-effect of chicory extracts of the invention on blood glucose in obese pre-diabetic mouse.

FIG. 7 shows the effect of a chicory extract low in chicoric acid, and of a chicory extract of the invention on blood glucose level in obese pre-diabetic mouse.

FIG. 8 shows the effect of purified chicoric acid on blood glucose level in obese pre-diabetic mouse.

FIG. 9 shows the effect of an Echinacea leaf extract, and of a chicory extract of the invention on blood glucose level in obese pre-diabetic mouse.

FIG. 10 shows the effect of an Echinacea leaf extract, and of a chicory extract of the invention on blood insulin and insulin resistance in obese pre-diabetic mouse.

### DETAILED DESCRIPTION OF THE INVENTION

The extract of a plant of the Cichorium genus of the invention may come from the following species, and mixtures thereof:

Cichorium intybus L.,

Cichorium intybus var. foliosum,

Cichorium intybus var. sativum,

Cichorium intybus ssp. intybus var. sativum, and

Cichorium endivia L.

The plant of the Cichorium genus is preferably chosen among the plants of the Cichorium intybus species, particularly the variety sativum and the Cichorium endivia species, and mixtures thereof.

In particular, the extract is obtained from the aerial part of the plant, and more particularly from the leaves. Thus the extract may be advantageously a leaf extract.

The expressions “weight of an extract of a plant of the Cichorium genus” or “weight of extract” in the sense of the invention mean the dry weight of the extract of a plant of the Cichorium genus or the dry weight of the extract.

The extract of a plant of the Cichorium genus according to the invention comprises phenolic acids derivatives and flavonoids derivatives. The phenolic acids derivatives comprise advantageously the three major phenolic acids derivatives identified as chicoric acid, caftaric acid and chlorogenic acid. The flavonoids are advantageously flavonoids of Quercetin, and Luteolin derivatives, particularly quercetin 3-O-glucuronide, isoquercitrin, and luteoline 7-O-glucuronide.

The amount of chicoric acid in the extract is advantageously of more or equal to 25 g/kg of extract, more particularly of more or equal to 30 g/kg of extract, and still more particularly of more or equal to 45 g/kg of extract. In particular embodiments, the amount of chicoric acid in the extract may be up to about 65 to 70 g/kg, depending mainly upon the chicory being used for preparing the extract. In general the amount of chicoric acid in the extract will be ranging from 45 to 55 g/kg.

The amount of caftaric acid in the extract is advantageously of more or equal to 2.5 g/kg of extract, more particularly of more or equal to 4 g/kg of extract, and still more particularly of more or equal to 5 g/kg of extract. In particular embodiments, the amount of caftaric acid in the extract may be up to about 10 g/kg, depending mainly upon the chicory being used for preparing the extract. In general the amount of caftaric acid in the extract will be ranging from 5 to 6 g/kg.

The amount of chlorogenic acid in the extract is advantageously of more or equal to 1 g/kg of extract, more particularly of more or equal to 1.5 g/kg of extract, and still more particularly of more or equal to 2 g/kg of extract. In particular embodiments, the amount of chlorogenic acid in the extract may be up to about 10 g/kg, depending mainly upon the chicory being used for preparing the extract. In general the amount of chlorogenic acid in the extract will be ranging from 5 to 6 g/kg.

The extract may comprise an amount of caftaric acid plus chicoric acid of more or equal to 30 g/kg of extract, more particularly of more or equal to 40 g/kg of extract, and still more particularly of more or equal to 50 g/kg of extract, preferably ranging from 50 to 60 g/kg.

The weight ratio chicoric acid/caftaric acid may range from 2 to 20, in particular from 3 to 15, more particularly from 4 to 14, and still more particularly from 6 to 12, advantageously from 7 to 11.

The weight ratio chicoric acid+caftaric acid/chlorogenic acid may range from 5 to 60, in particular from 6 to 40, more particularly from 7 to 50, and still more particularly from 8 to 20, advantageously from 9 to 19.

The amount of flavonoids derivatives in the extract, expressed in terms of the corresponding standard of quercetin, is advantageously of more or equal to 4.5 g/kg of extract, more particularly of more or equal to 5 g/kg of extract, and still more particularly of more or equal to 6.5 g/kg of extract, preferably ranging from 6.5 to 10 g/kg.

The amount of flavonoids may be determined using quercetin as standard, based on its UV spectrum. In other word, the amount of flavonoids is the weight of flavonoids, in particular quercetin derivatives, expressed in terms of the corresponding standard of quercetin.

The extract advantageously comprises a total phenolic content ranging from 3 to 10% by weight total phenol content equivalent to gallic acid.

The total phenolic content in the extract may be determined according to the method using Folin-Ciocalteu reagent and/or also by reversed phase HPLC.

The method of preparing the extract according to the invention from the Cichorium genus raw material has to follow a process adapted to result in an active extract comprising the phenolic acids derivatives and the flavonoids derivatives as defined above and in the examples.

Particularly, the method has to take into consideration the stability of the phenolic acids derivatives and the flavonoids derivatives. Purification of the extract has to be performed taking into account the sensitivity of the phenol derivatives to certain reactants such as polyphenols oxidases, since phenolic acids derivatives and flavonoid derivatives are sensitive to said enzymes. The person skilled in the art shall identify such components to be avoided in the extraction and purification process, based on his own skills and knowledges and based on the disclosure of the specific embodiments of the invention.

The extract according to the invention is advantageously an aqueous or hydro-alcoholic extract, in particular an hydro-ethanolic extract. The extract may in particular be the extract obtained or obtainable via the method disclosed in this description.

The invention also concerns a composition comprising an extract of a plant of the Cichorium genus, such as disclosed above and below, and optionally a carrier.

The carrier may particularly be an edible carrier usually used for the preparation of pharmaceutical compositions, neutraceutical compositions and dietary or food supplements. The carrier may comprise one or more flavour agents.

The composition may comprise an amount of an extract of a plant of the Cichorium genus going from 10 to 99% by weight, more particularly from 25 to 95% by weight, even more particularly from 50 to 95% by weight, still more particularly from 75 to 95% by weight compared to the total weight of the composition.

The dietary or food supplement may comprise an amount of extract ranging from 1 to 100% by weight compared to the total weight of the supplement.

The nutraceutical or the food composition may comprise an amount of extract ranging from 0.1 to 5% by weight compared to the total weight of the composition.

The supplements or the nutraceutical or food composition may be a liquid, a solid or a powder.

The composition may be formulated in order to allow a daily uptake for humans ranging from 5 to 60 mg/kg, and in particular around 30 mg/kg.

The invention also concerns a method for preparing an extract of a plant of the Cichorium genus as defined above and below, which extraction process comprises:
a) contacting crushed Cichorium genus plant(s) with an extraction solvent,
b) filtering out the solids and collecting the solvent extract,
c) optionally washing the solids by stirring with an extraction solvent
d) optionally filtering the solids and collecting the solvent extract,
e) combining the solvent extracts and removing non-soluble residues,
f) concentrating the solvents,
g) optionally enriching the phenolic compounds comprising the extract, in particular by precipitation using food grade alcohol,
h) filtering and evaporating the filtrate, and
i) recovering the dry extract.

The extraction process of the invention is preferably conducted with the aerial parts of the plants, and more preferably with the leaves.

The plant may be used fresh after being blanched or may be used dried. Preferably dried or blanched fresh leaves of a plant of the Cichorium genus are used in step a.

Extraction in step a is advantageously performed at a temperature above 25° C., particularly above 35° C., more particularly above or equal to 50° C.

In particular, the extraction in step a is advantageously performed at a temperature above 25° C., more particularly above 35° C., and still more particularly of around 50° C.

Advantageously, the extraction in step a is advantageously performed at a temperature between 25° C. and 70° C., preferably between 40° C. and 60° C.

The solvent of the extraction process are preferably chosen among aqueous, alcoholic, organic solvents soluble in water and combinations thereof. Among the organic solvents soluble in water may be cited acetone.

The extract being obtain may be qualified to be aqueous, alcoholic, or organic solvent extracts.

Suitable solvents may be chosen from water, methanol, ethanol, acetone, n-propanol, iso-propanol, 2-butanol, and combinations thereof.

More particularly the extraction solvent comprises at least 75%, at least 90%, more particularly at least 95%, and even more particularly at least 99% by weight of organic solvent compared to the total weight of the extraction solvent, and still more particularly consists of such solvent(s).

Following an embodiment, the extraction is performed with water and/or an alcoholic solvent, in particular ethanol.

In case of an extraction performed with water and alcohol, in particular ethanol, the volume ratio water/alcohol may range from 80/20 to 20/80, in particular form 70/30, to 30/70, more particularly from 60/40 to 40/60, and even more particularly is of around 50/50.

The percentage of alcohol, and in particular ethanol, used for extraction can have an impact on the yield and composition of the biologically active compounds.

The extraction may be done by batch or continuously, or by successive uses of a same extraction solvent in successive batches to obtain saturation of the solvent with the extract. For batch extraction, the person skilled in the art shall define appropriate weight ratio solvent/solid parts of the plant, optimized for an industrial process. The ratio solvent/solid ratio may be ranging from 2 to 20, in particular 5 to 15 and more particularly around 10 times the weight of solids. The extraction may be done one or several time, in particular 2 to 4 times.

Low ratios may be used in continuous or successive batch extraction processes.

The method of the invention may be performed at a temperature ranging from 25 to 70° C., in particular from 40 to 60° C., and more particularly around 50° C.

The extraction may last from 1 to 5 hours, in particular around 2 hours, under stirring, for example mechanical or magnetic stirring.

The remaining solids may be filtered out, in particular through a filter bag.

Several extracts may be combined to form a single extract according to the invention.

The wet solids may be extracted another time by stirring with a solvent, in particular a hydro-alcoholic mixture, with a volume from 1 to 100 times the weight of the dry solids, and a stirring for about 10 to120 minutes further.

The solids are collected and the extracts may be combined.

The different extraction solutions may be combined and left for decantation, filtered through filter paper or centrifugation to remove non-soluble residues.

The clear supernatants obtained may be concentrated to about 5% to 20% of their initial volume, for example using a concentrator, and may be then treated with food grade alcohol, in particular ethanol, in a definite proportion, for example a minimum of 2 times the concentrated volume, to remove any precipitate formed. This step may allow removing all or parts of undesired compounds, such as polysaccharides or water soluble proteins.

A powdered extract may be obtained by drying the concentrated extracts, for example using spray drier, oven at 50-80° C., or vacuum drier.

The dry extract is weighed (g) and the extraction yield is calculated by the formula:

Dry Yield %=(weight of dry extract/weight dry plant)×100

Fresh yield %=(weight of dry extract/weight of fresh raw plant material)×100

More particularly, the plant extract may be obtained by the process comprising the following steps:
a) contacting crushed and pressed plant(s) with 2 to 20 times, in particular around 10 times, their weight with an extraction solvent, in particular a mixture ethanol-water comprising at least 30% by volume of ethanol, and more particularly a mixture ethanol-water having a 1/1 volume ratio, and stirring, for example for 2 hours, in particular at a temperature above 25° C., more particularly above 35° C., and still more particularly of around 50° C.,
b) filtering out the solids and collecting the solvent extract,
c) washing the wet solid by stirring with an extraction solvent, in particular with a volume corresponding to 1 to 100 times the weight of the dry solids, for example for about 15 to 120 minutes, particularly for 15 to 30 minutes.
d) filtering the solids and collecting the solvent extract,
e) combining the extraction solutions and decanting or filtering them to remove non-soluble residues,
f) concentrating the solvents, and enriching the extract by precipitating in ethanol
g) concentrating the filtrate under reduced pressure, and
h) drying, for example by staying in an oven at 50-80° C. under vacuum, for example with vacuum spray drying, or the concentrate may be freezed for lyophilisation, and
i) recovering the dry extract.

In general, the yield of extraction is ranging from 1.5 to 3.5% by weight of extract compared to the total weight of freshly crushed cake. The yield is ranging from 12 to 17% when using dried leaves.

The plant extract can be prepared on a commercial scale by repeating the extraction process that lead to the isolation of the extract of interest.

Thus small-scale extraction procedure can be scaled up, with optionally additional steps of quality control included to ensure reproducible results for the resulting extracts.

Various extraction processes can be employed. Generally, the extract is obtained by contacting the solid plant(s) with a solvent with adequate mixing and for a period of time sufficient to ensure adequate exposure of the solid plant material to the solvent such that biologically active molecules present in the plant material can be taken up by the solvent.

The solvent extraction process may be selected from direct and continuous (counter-current) extraction types at room temperature or at higher temperature with polar and/or non-polar solvent(s). Adequate contact of the solvent with the plant material can be encouraged by shaking the suspension. The liquid fraction is then separated from the solid (insoluble) matter resulting in the generation of two fractions: a liquid fraction, which is the potential extract, and a solid fraction. Separation of the liquid and solid fractions can be achieved by one or more standard processes known to those skilled in art.

The invention also concerns an extract susceptible to be obtained or obtainable by the process of the invention, said extract comprising phenolic acids derivatives and flavonoids derivatives.

### EXAMPLES

### Example 1

### Preparation of Cichorium Intybus L Extracts

### Materials and Methods

Dry aerial parts (100 g) of Cichorium intybus L. are finely chopped (5-8 mm), before extraction.

### Extraction Process

The plant is extracted with about 1000 ml of a 50% ethanol/water (V/V) for 2 hours at 50° C., under mechanical agitation. At the end of the extraction period, the solids are filtered out through a filter bag (PE-100) or other similar straining material.

The wet solids are extracted one more time by stirring with another 3 volumes of ethanol 50% for about 15 to 30 min further.

The solids are again filtered out and the obtained extracts were combined together and 980 ml of the liquid extract is obtained, left for decantation and filtered through filter paper or centrifugation to remove non-soluble residues.

The extraction solution was concentrated under reduced pressure to 5-20% of its initial volume and then enriched by precipitating in food grade ethanol. The filtrate is again concentrated and set to dryness. The dry extract is recovered.

The HPLC chromatograms of the plant extracts at 280 nm of Example 1 is shown in FIG. 1 as Chromatogram I, wherein A refers to Caftaric acid, B to Chlorogenic acid, C to Chicoric acid, D to Quercetin 3-O-glucuronide and Isoquercitrin, and E to Luteolin 7-O-glucuronide. Furthermore, the composition of the extract is shown in Table 1 below.

### Example 2

### Preparation of Cichorium Intybus Ssp. Intybus Var. Sativum Extracts

The same procedure than Example 1 was followed except that fresh Cichorium intybus ssp. intybus var. sativum aerial parts was used and blanched before the chopping step.

The HPLC chromatograms of the plant extracts at 280 nm of Example 2 is shown in FIG. 1 as Chromatogram II wherein A refers to Caftaric acid, B to Chlorogenic acid C to Chicoric acid, D to Quercetin 3-O-glucuronide and Isoquercitrin, and E to Luteolin 7-O-glucuronide. Furthermore, the composition of the extract is shown in Table 1 below.

### Example 3

### Composition of the Extracts from Examples 1 and 2

### Analytical Methods

The extracts from examples 1 and 2 comprised between 3 and 7% by weight total phenol content equivalent to gallic acid, using Folin-Ciocalteu method.

### Characterization and Quantification of the Polyphenol Content by HPLC:

### HPLC-DAD Analysis:

Analytical HPLC (Dionex) is set up as required. In this invention, the mobile phase used was 0.1% formic acid into 1000 ml high purity water (solvent A) and acetonitrile (solvent B), utilizing the following gradient over a total run of 96 minutes with a flow rate of 0.8 ml/min. The gradients points were for time 0.0 minutes-95% A and 5% B; for time 10 minutes-90% A and 10% B; held isocratic for 10 minutes and from 20 minutes to 40 minutes the gradient varied linearly from 10% to 20% B; again held isocratic for 10 minutes; from 50 minutes to 65 minutes the gradient varied linearly from 20% to 30% B and to 50% during the next 10 minutes; from 75 minutes to 76 minutes the gradient varied linearly from 50% to 100% B and was held isocratic for 10 minutes. Back to original conditions of 95% A and 5% B from 85 minutes to 86 minutes and held isocratic for 10 minutes. Phenolic compounds in the eluent were detected with a UV-diode-array set at 280 nm using a reversed phase C-18 column (250×4.6 mm ID×5 μm; ACE). The amounts of phenolic acids in the extracts were determined using calibration curves of: Caftaric acid, Chlorogenic acid, Chicoric acid and the amounts of flavonoids in the extracts were determined using calibration curves of: Quercetin.

### HPLC-DAD/ESI-MS Analysis:

HPLC/MS analysis of the extract was performed using an HPLC (Thermo Finnigan surveyor), and interfaced to an LCQ ion trap spectrometer fitted with an electrospray interface (Thermo Finnigan, LCQ Advantage max). The elution program was the same as above, and the experiment was performed in both negative and positive modes. Spectra were scanned over a mass range of m/z 80-2000.

TABLE 1

| | Phenolic acids | | | Flavonoids | |
|---|---|---|---|---|---|
| | derivatives | | | derivatives | Total |
| | Caftaric | Chlorogenic | Chicoric | content | polyphenol |
| | acid (A) | acid (B) | acid (C) | (D-E) | content (g |
| Plant | (g/kg | (g/kg | (g/kg | (g Quercetin/ | Gallic acid/ |
| extracts | extract) | extract) | extract) | kg extract | kg extract) |
| Ex. 1 | 5.01 g | 6.5 g | 54.52 g | 6.97 g | 69 g |
| Ex. 2 | 5.97 g | 2.79 g | 46.82 g | 9.91 g | 54 g |

Quantitative composition of extracts from Examples 1 and 2 by HPLC at 280 nm

### Example 4

### Effect of Chicory Leaf Extract (CLE) on Blood Glucose (Glycemia), Blood Insulin (Insulinemia), Insulin Resistance, Body Weight, Fat Storage and Fatty Liver

### Materials and Methods

Five-week old C57BL/6NCrI mice (Charles River Laboratories, France) weighing around 20 g were used for the experiment. After 8 days of acclimation, mice were randomly assigned to the different experimental groups (9 animals per group) according to their fasted glycemia.

On Day 0, one group was kept under normal diet (23% of calories from proteins, 66% from carbohydrates, and 11% from fat) and the others were submitted to high fat diet (17% of calories from proteins, 28% from carbohydrates, and 55% from fat). The diets and water were provided ad libitum. The dosing formulations were administered by oral gavage every morning between 9 h and 10 h from Day 1 to Day 56. The dose volume was 10 ml/kg of body weight. The actual volume administered was calculated and adjusted based on the most recent body weight of each animal. The conditions tested were the followings:
Control Normal: Mice were fed a normal diet and were administered water once daily.
Control High Fat: Mice were fed a high fat diet and were administered water once daily.
CLE 400 mg/kg: Mice were fed a high fat diet and were administered Chicory Leaf extract (CLE) once daily at the dose of 400 mg/kg of body weight.

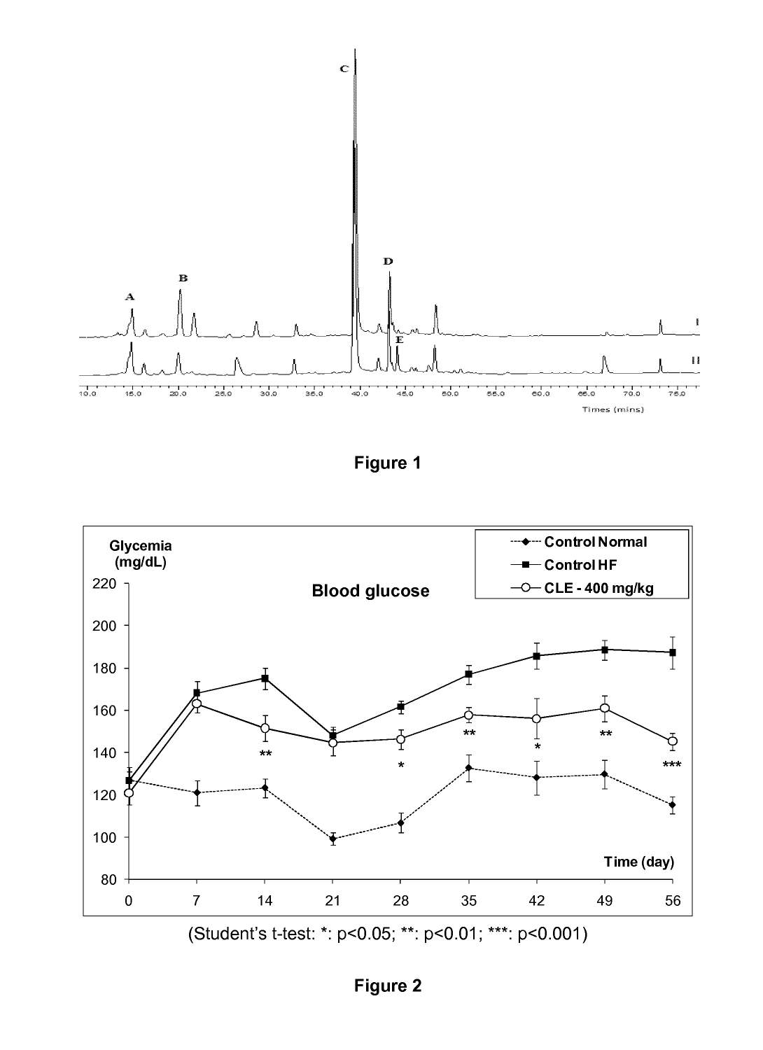

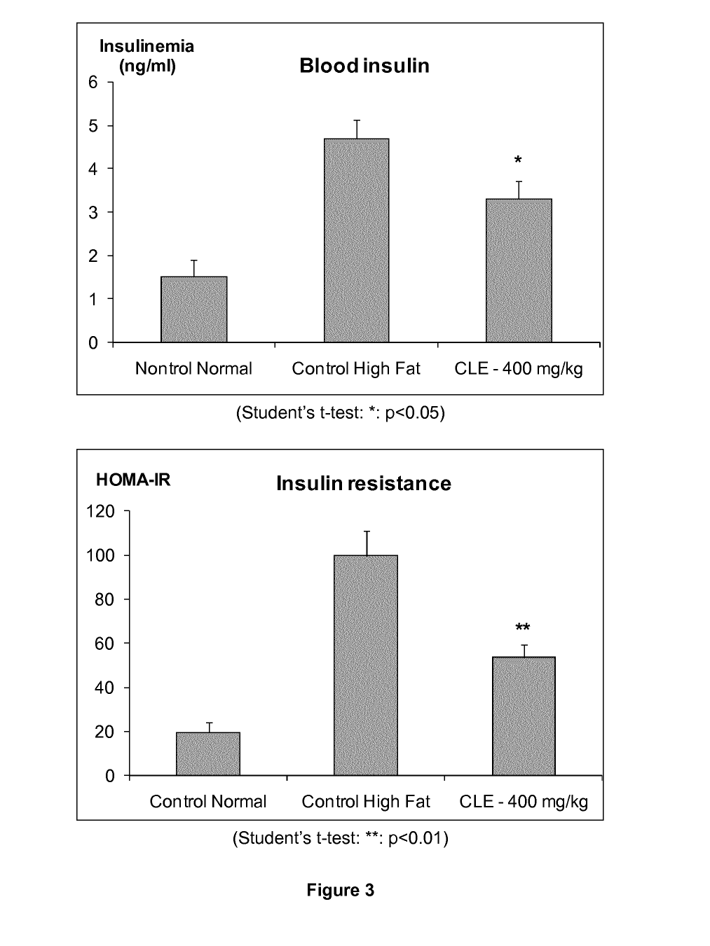

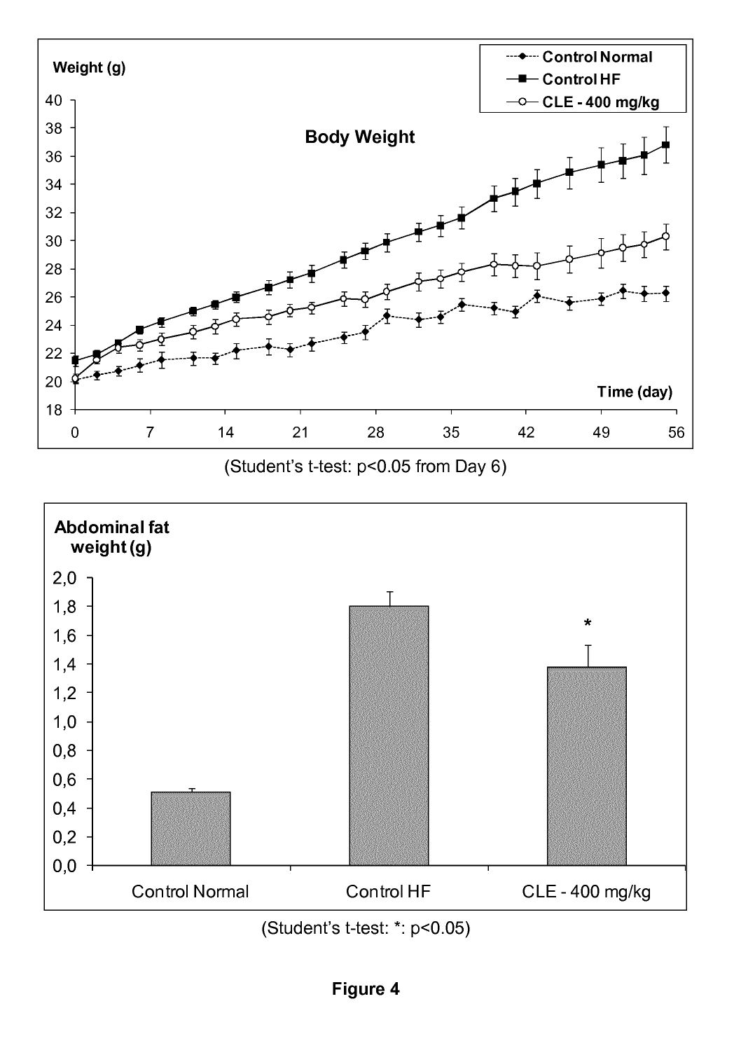

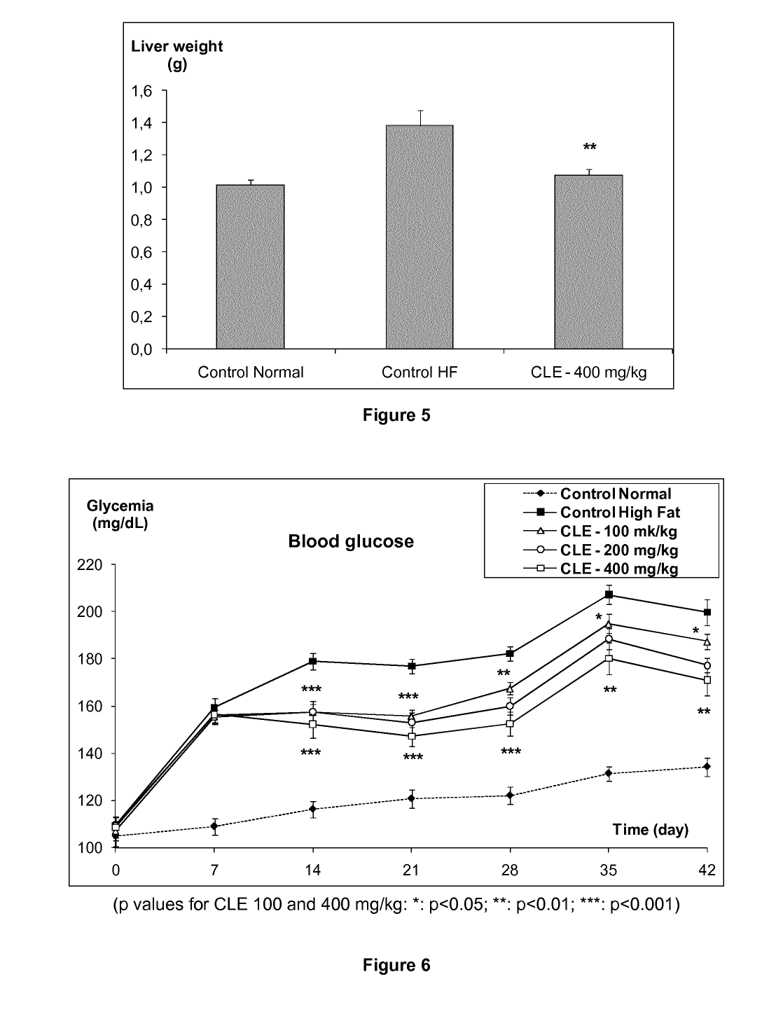

The effect of CLE on glycemia is shown on FIG. 2, on insulin and insulin resistance on FIG. 3, on body weight and fat storage on FIG. 4, and on fatty liver on FIG. 5.

Body weights of the mice were recorded at the arrival and then 3 times a week (on Monday, Wednesday, and Friday).

Fasting blood glucose levels were measured the day of randomization (Day 0) and on Day 7, 14, 21, 28, 35, 42, 49, and 56 between 13h00 and 14h00 on animals fasted for 4 hours. Whole blood samples (one drop) were collected via the tail vein for glucose determination using a hand-held glucometer (OneTouch Ultra 2, LifeScan).

At the end of the study, after a 4 hour fast, animals were anaesthetized by an intra peritoneal injection of 0.1 ml of Pentobarbital Sodium. A terminal blood sample was collected via cardiac puncture using heparin as anticoagulant. This terminal blood sampling conducts to the death of the animals. Blood samples were put at 4° C., centrifuged in the 30 min after collection; plasma harvested and kept frozen pending insulin analysis. The epididymal fat pad (abdominal fat) and the liver were harvested to measure their weights.

Fasting plasma insulin levels were measured using an ELISA kit (Mercodia, Sweden). Then, insulin resistance indexes (HOMA-IR) were calculated using the formula: HOMA-IR=fasting insulin (mU/l)×fasting glucose (mmol/l)/22.5.

This protocol was approved by the Regional Ethic Committee (Montpellier, France).

### Results

### Blood Glucose (FIG. 2)

After one week under diets (Day 7), fasting glycemia of mice submitted to high fat diet was significantly higher (168±5.7 mg/dL) than those of mice submitted to normal diet (121±5.9 mg/dL). Thereafter, this difference stayed rather constant all over the study.

Chicory leaf extract reduced the glycemia of the pre-diabetic mice. At the end of the study (Day 56), glycemia was reduced by 58%. This effect is clearly significant at Day 14, 28, 35, 42, 49, and 56.

### Blood Insulin and Insulin Resistance (FIG. 3)

Fasting insulinemia was also significantly increased by high fat diet, reaching 4.70±0.43 ng/ml compared to 1.53±0.37 ng/ml for mice under normal diet. In agreement with these increases in fasting glycemia and insulinemia induced by high fat diet, insulin resistance index (HOMA-IR) was multiplied by 5 showing that these animals are strongly insulin resistant.

At the end of the treatment period (Day 56), CLE significantly reduced fasting blood insulin level induced by high fat diet (p<0.05). Insulinemia reached 3.31±0.40 ng/ml for CLE treated mice. Therefore, according to the HOMA-IR insulin resistance index, CLE reduced by 58% insulin resistance induced by high fat diet in pre-diabetic mice (p<0.01).

### Body Weight and Abdominal Fat Weight (FIG. 4)

Mice submitted to high fat diet gained much more weight than those under normal diet. At the end of the study, after 8 weeks of diet, mice under high fat diet gained 2.5 times more weight than the control mice under regular diet. They reached 36.8±1.3 g, whereas mice under normal diet reached 26.3±0.5 g.

This increase of body weight induced by the diet is notably due to the storage of the energy in form of abdominal fat. Indeed, epididymal (abdominal) fat weights of mice under high fat diet were multiplied by 3.5 during the study compared to those of mice under normal diet: 1.80±0.11 and 0.51±0.03 g for high fat and normal diet, respectively. Perivisceral fat is recognized to be associated with or to be a strong risk factor of insulin resistance and metabolic syndrome.

CLE at the dose of 400 mg/kg of body weight reduced body weight gain induced by high fat diet. This effect is significant from Day 4 of treatment and last all along the study (p<0.01 from Day 6). At the end of the study, mice under high fat diet treated with CLE gained 10.1±0.8 g, whereas mice under high fat diet treated with water gained 15.4±1.3 g and mice under normal diet treated with water gained 6.1±0.4 g.

This reduction is at least partly due to a reduction of fat storage in abdominal fat. CLE reduced by 33% fat storage into epididymal fat in mice submitted to high fat diet (p<0.05). Epididymal fat weight of CLE treated mice was 1.38±0.15 g, compared to 1.80±0.11 and 0.51±0.03 g for high fat diet control mice and normal diet control mice, respectively.

### Liver Weight (FIG. 5)

High fat diet induced the storage of fat into the liver: what is called fatty liver or hepatic steatosis. The color of the liver became white instead of red for mice under normal diet and its weight was increased by 35%: 1.38±0.10 g for high fat diet versus 1.02±0.03 g for normal diet.

CLE almost normalized fatty liver that is reduced by 83% (p<0.01).

### Conclusions

HDF induce a clear phenotype of metabolic syndrome in C57BL/6 mice with a significant increase in body weight gain, abdominal fat storage, fatty liver, fasting glycemia and insulinemia, and insulin resistance.

Chicory leaf extract at the dose of 400 mg/kg of body weight markedly reduced the defects induced by chronic consumption of high fat diet in mice. That comprises:
Reduction of fasting glycemia
Reduction of fasting insulinemia
Reduction of insulin resistance
Reduction of body weight gain
Reduction of abdominal fat storage
Reduction of hepatic steatosis

Therefore, chicory leaf extract is shown to be a good health good ingredient to address one or several metabolic syndrome(s) and related defects.

### Example 5

### Dose-Effect of Chicory Leaf Extracts (CLE) on Blood Glucose (Glycemia)

### Materials and Methods

The protocol was the same than for Example 4.

The conditions tested were the followings:
Control Normal: Mice were fed a normal diet and were administered water once daily.
Control High Fat: Mice were fed a high fat diet and were administered water once daily.
Chicory leaf extract (CLE) 100, 200, or 400 mg/kg: Mice were fed a high fat diet and were administered CLE once daily at the dose of 100, 200, or 400 mg/kg of body weight.

### Results

### Blood Glucose (FIG. 6)

After one week under diets (Day 7), fasting glycemia of mice submitted to high fat diet was significantly higher (159±3.9 mg/dL) than those of mice submitted to normal diet (109±3.6 mg/dL). Thereafter, this difference stayed rather constant all over the study.

Chicory leaf extract reduced the glycemia of the pre-diabetic mice from Day 14 to Day 42. This effect increased with the dose of CLE and is significant all over the study, for the 3 doses. At the end of the study, the glycemia reached 187±3.2, 177±3.1, 171±6.3 mg/dL for 100, 200, and 400 mg/kg, respectively, compared to 200±5.4 mg/dL for the control.

### Conclusions

Chicory leaf extract (CLE) markedly reduced fasting hyperglycemia induced by chronic consumption of high fat diet in mice in a dose-dependent manner. This effect is already significant at the lower dose tested: 100 mg/kg of body weight.

### Example 6

### Chicory Leaf Extracts (CLE) Containing Low Levels of Chicoric Acid are Less Effective to Reduce Blood Glucose

Materials and Methods

Low Chicoric Acid-CLE—Extraction Process

Low chicoric acid-CLE was extracted following the extraction process presented in Example 2 except that the blanching step before chopping was not done. Consequently, the polyphenol oxidases of the plant were not inactivated leading to lower level of polyphenols. Low chicoric acid-CLE contained 1.05% of chicoric acid.

CLE—Extraction Process

CLE was extracted following the extraction process presented in Example 2. CLE contained 4.68% of chicoric acid.

Pharmacological Protocol

The protocol is comparable to Example 4 but the C57BL/6 mice were submitted to high fat diet for 1 week to induce pre-diabetes before the onset of the treatment. Then, CLE and Low chicoric acid-CLE were given for 3 weeks by oral gavage at the dose of 200 mg/kg of body weight.

Results

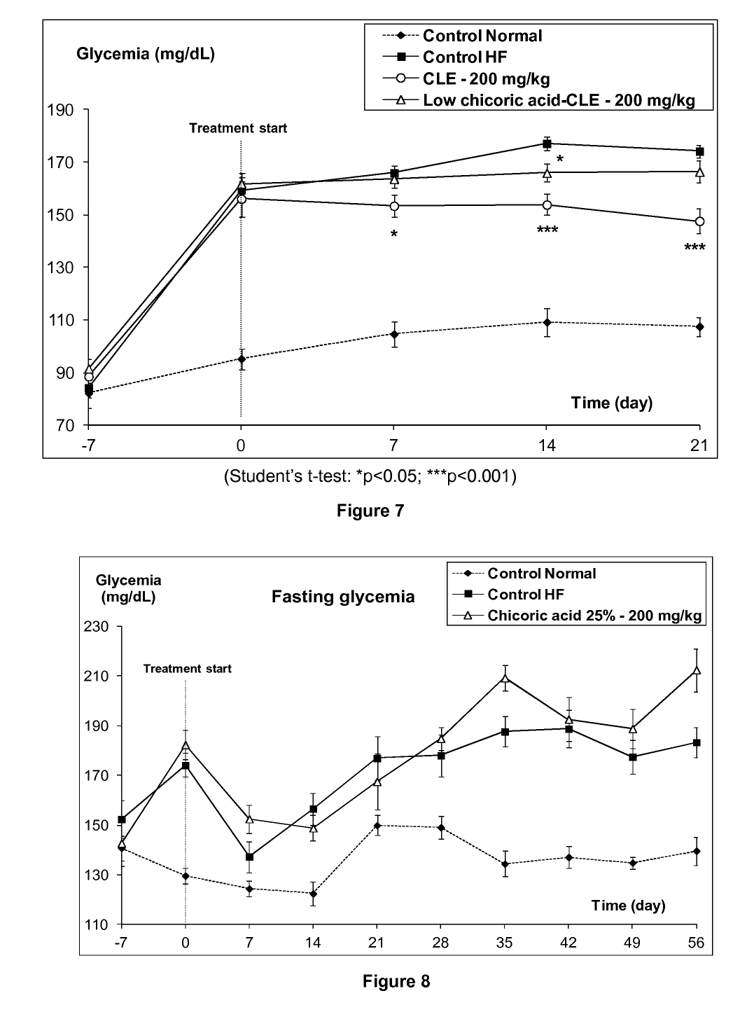

Effect on Fasting Glycemia (FIG. 7)

After one week under diets, fasting glycemia of mice submitted to high fat diet (159.6±4.9 mg/dL) was significantly higher (p<0.001) than those of mice submitted to normal diet (95.2±4 mg/dL). These results confirm the fact that mice under high fat diet were hyperglycemic at the onset of the treatment.

CLE reduced glycemia of the pre-diabetic mice by 31.5% in mean (% of reduction to come back to normal values observed with Control Normal). This effect was clearly significant from the first week of treatment and lasted up to the end of the study (see FIG. 7: *p<0.05 at Day 7 and ***p<0.001 at Day 14 and 21).

By contrast, Low chicoric acid-CLE presented a much lower effect. It reduced pre-diabetic mice glycemia by 10.6% in mean. This effect was significant at Day 14 (see FIG. 7: *p<0.05) but not at Day 7 and 21.

Conclusions

C57BL/6 mice submitted to high fat diet for 1 week became hyperglycemic. Chicory leaf extract (CLE; containing 4.68% of chicoric acid) at the dose of 200 mg/kg of body weight markedly reduced hyperglycemia and this effect is linked to the presence of sufficient amount of chicoric acid in the extract as Low chicoric acid-CLE (containing 1.05% of chicoric acid) was poorly efficient.

Therefore, chicoric acid is one of the components of CLE that is important for the efficacy of the extract. A sufficient amount of chicoric acid in the extract is necessary to observe an effect on glycemia reduction.

### Example 7

### Purified Chicoric Acid (25%) is not Effective to Reduce Blood Glucose

Materials and Methods

Purified Chicoric Acid (25%)

Purified chicoric acid was obtained from a commercial source. Briefly, polyphenols extracted from a vegetal source were purified on an adsorbent resin leading to a product rich in polyphenols and containing around 25% of chicoric acid.

Pharmacological Protocol

The protocol is comparable to Example 4 but the C57BL/6 mice were submitted to high fat diet for 1 week to induce hyperglycemia before the onset of the treatment. Then, purified chicoric acid at 25% was given for 8 weeks by oral gavage at the dose of 200 mg/kg of body weight.

Results

Effect on Fasting Glycemia (FIG. 8)

At the difference with CLE, purified chicoric acid (25%) presented no effect on fasting glycemia of the pre-diabetic mice even after 8 weeks of treatment (FIG. 8).

Conclusions

Therefore, even if chicoric acid is an essential component for CLE efficacy, it is not sufficient. Co-factors, which are suppressed in chicoric acid purification, are also important for the effect of CLE on the reduction of fasting glycemia in pre-diabetic mice.

### Example 8

### Chicory Leaf Extracts (CLE) is More Potent than an Echinacea Leaf Extract (EchLE)

Materials and Methods

Echinacea Leaf Extract (EchLE)—Extraction Process

EchLE was extracted following the extraction process presented in Example 1. EchLE contained 3.32% of chicoric acid.

Chicory Leaf Extract (CLE)—Extraction Process

CLE was extracted following the extraction process presented in Example 2. CLE contained 4.68% of chicoric acid.

Pharmacological Protocol

The protocol is identical to Example 6. C57BL/6 mice were submitted to high fat diet for 1 week to induce pre-diabetes before the onset of the treatment. Then, CLE and EchLE were given for 3 weeks by oral gavage at the dose of 200 mg/kg of body weight.

Results

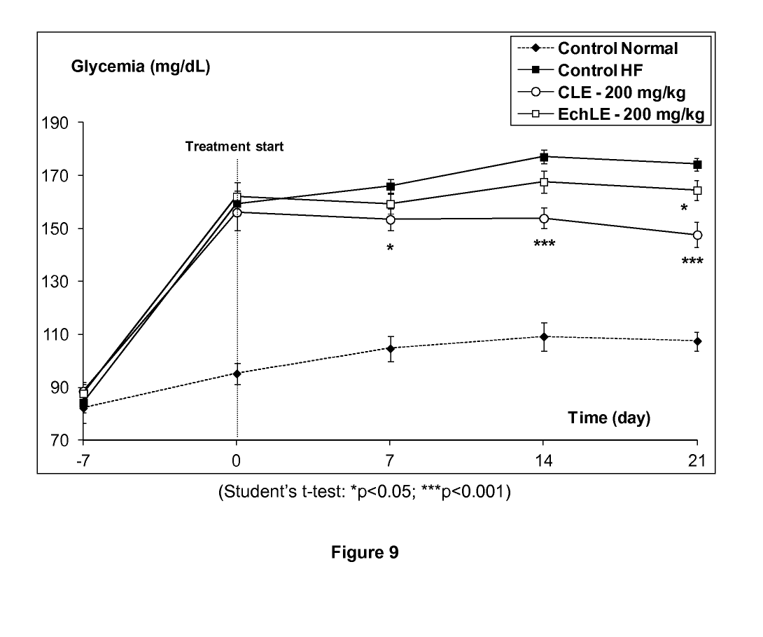

Effect on Fasting Glycemia (FIG. 9)

CLE extract reduced glycemia of the pre-diabetic mice by 31.5% in mean (% of reduction to come back to normal values observed with Control Normal). This effect was clearly significant from the first week of treatment and lasted up to the end of the study (see FIG. 9: *p<0.05 at Day 7 and ***p<0.001 at Day 14 and 21).

EchLE presented a much lower effect. It reduced pre-diabetic mice glycemia by 13.1% in mean. This effect was significant at Day 21 (see FIG. 9: *p<0.05) but not at Day 7 and 14.

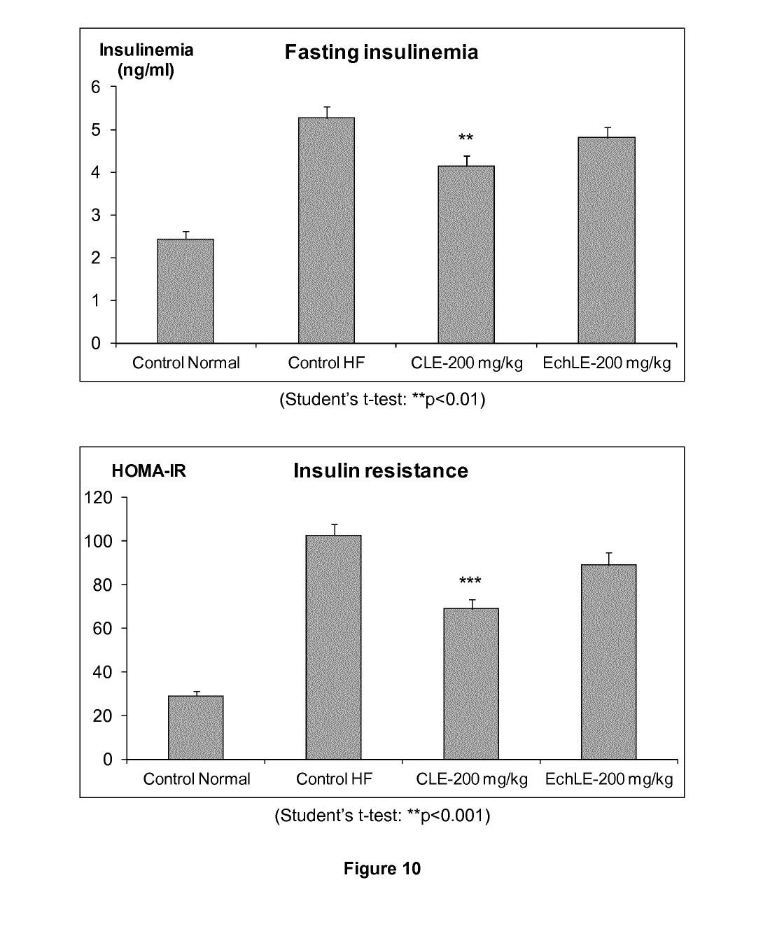

Blood Insulin and Insulin Resistance (FIG. 10)

At the end of the treatment period (Day 21), fasting insulinemia was also significantly increased by high fat diet, reaching 5.26±0.27 ng/ml compared to 2.43±0.20 ng/ml for mice under normal diet. In agreement with these increases in fasting glycemia and insulinemia, insulin resistance index (HOMA-IR) was multiplied by 3, showing that the mice were clearly insulin resistant at the end of the protocol.

CLE significantly reduced fasting blood insulin level induced by high fat diet to 4.16±0.23 ng/ml (FIG. 10: **p<0.01). Therefore, according to the HOMA-IR insulin resistance index, CLE reduced by 45.9% insulin resistance induced by high fat diet in pre-diabetic mice (FIG. 10: ***p<0.001).

In the same condition, EchLE slightly reduced insulinemia to 4.16±0.24 ng/ml but this effect is not statistically significant. Also, insulin resistance was non-significantly decreased by 18.7% (FIG. 10).

Conclusions

Chicory leaf extract (CLE; containing 4.68% of chicoric acid), at the dose of 200 mg/kg of body weight, markedly reduced hyperglycemia, hyperinsulinemia, and insulin resistance of pre-diabetic mice. These effects are linked to the presence of sufficient amount of chicoric acid in the extract but also to the chicory origin of the extract as Echinacea leaf extract (EchLE), containing quite high amount of chicoric acid (3.32% of chicoric acid), is much less efficient. Co-factors, present in Cichorium genus but not in Echinacea genus, are important for the effects of CLE.

Taking together, these results show that chicoric acid and co-factors in the extract coming from Cichorium genus are important for the efficacy of CLE.

1-20. (canceled)

21. An extract of a plant of the Cichorium genus comprising chicoric acid, caftaric acid and chlorogenic acid and Quercetin and/or Luteolin derivatives, wherein the amount of chicoric acid is of more or equal to 25 g/kg of extract, and optionally a carrier.

22. The extract of claim 21, wherein the amount of caftaric acid is of more or equal to 2.5 g/kg of extract.

23. The extract of claim 21, wherein the amount of chlorogenic acid is of more or equal to 1 g kg of extract.

24. The extract of claim 21, wherein the amount of flavonoids derivatives in the extract, expressed in terms of the corresponding standard of quercetin, is of more or equal to 4.5 g/kg of extract.

25. A method for the preparation of the extract according to claim 21, comprising
a) contacting crushed Cichorium genus plant(s) with an extraction solvent,
b) filtering out the solids and collecting the solvent extract,
c) optionally washing the solids by stirring with an extraction solvent
d) optionally filtering the solids and collecting the solvent extract,
e) combining the solvent extracts and removing non-soluble residues,
f) concentrating the solvents,
g) optionally enriching the phenolic compounds comprising the extract, in particular by precipitation using food grade alcohol,
h) filtering and evaporating the filtrate, and
i) recovering the dry extract;
wherein the method is performed at a temperature ranging from 40 to 60° C.

26. The method of claim 25, wherein the plant of the Cichorium genus is chosen among the group consisting of plants of the Cichorium intybus species, plants of the Cichorium endivia species, and mixtures thereof.

27. The method of claim 25, wherein aerial parts of the plant are used.

28. The method of claim 25, wherein the plant is dried or is fresh and blanched.

29. The method of claim 25, wherein the extraction solvent comprise water and an alcohol in a volume ratio water/alcohol ranging from 80/20 to 20/80.

30. A composition comprising the extract of a plant of the Cichorium genus comprising phenolic acids derivatives and flavonoids derivatives according to claim 21, and optionally a carrier.

31. The extract of claim 21 for a use selected in the group consisting of:
use in a curative or prophylactic treatment of control of body weight, and/or of weight gain.
use in a curative or prophylactic treatment of increased fat storage, and/or of fatty liver
use in a curative or prophylactic treatment of high liver triglycerides level
use in a curative or prophylactic treatment of hypertriglyceridemia
use in a curative or prophylactic treatment of hyperglycemia
use in a curative or prophylactic treatment of hyperinsulinemia
use in a curative or prophylactic treatment of insulin resistance
use in a curative or prophylactic treatment of pre-diabetes
use in a curative or prophylactic treatment of metabolic syndrome.

32. A dietary or food supplement, nutraceutical or food composition comprising the extract according to claim 21, and optionally a carrier.
